(19) United States
(12) Patent Application Publication
(10) Pub. No.: US 2025/0387155 A1
(43) Pub. Date: Dec. 25, 2025

(54) LOW PROFILE ELECTRODES FOR A SHOCK WAVE CATHETER

(71) Applicant: Shockwave Medical, Inc., Santa Clara, CA (US)

(72) Inventors: Khanh VO, Daly City, CA (US); Jonathan GALLEGO, San Jose, CA (US); Huy PHAN, Santa Clara, CA (US)

(73) Assignee: Shockwave Medical, Inc., Santa Clara, CA (US)

(21) Appl. No.: 19/315,436

(22) Filed: Aug. 29, 2025

Related U.S. Application Data
(62) Division of application No. 16/993,114, filed on Aug. 13, 2020, now Pat. No. 12,426,938.
(60) Provisional application No. 62/904,839, filed on Sep. 24, 2019.

(51) Int. Cl. A61B 18/14; A61B 17/22; A61B 18/00; A61B 18/12

(52) U.S. Cl. CPC A61B 18/14; A61B 18/1206; A61B 2017/22025; A61B 2018/00077; A61B 2018/00083; A61B 2018/0022; A61B 2018/00422; A61B 2018/1467

(57) ABSTRACT

The present invention provides a system and method for treating tight, hard-to-cross calcified lesions in which an angioplasty balloon is used to dilate the lesions and provide shock waves to restore normal blood flow in a patient's artery. An exemplary device includes an elongated tube and a balloon wrapped circumferentially around the tube and sealed to a distal end of the tube. During treatment, the device is advanced into a patient's vasculature and the balloon is inflated with conductive fluid such that the balloon is fixed to walls of the vasculature proximal to the calcified lesion. The balloon includes at least one low-profile emitter positioned near the distal end of the balloon, which may be activated to generate shock waves to break loose calcifications in the lesion. After calcium in the tight lesion has been modified, the balloon can be deflated and advanced further into the lesion to continue treatment.

### CROSS-REFERENCE TO RELATED APPLICATIONS

This application is a divisional of U.S. patent application Ser. No. 16/993,114, filed on Aug. 13, 2020, which claims priority to U.S. Provisional Patent Application No. 62/904,839, entitled “LOW PROFILE ELECTRODES FOR A SHOCK WAVE CATHETER,” filed on Sep. 24, 2019, the content of each of which is hereby incorporated by reference in its entirety.

### FIELD OF THE DISCLOSURE

The present disclosure relates generally to shock wave electrodes, and more specifically, to electrodes for the generation of shock waves within vascular structures.

### BACKGROUND

The present invention relates to a treatment system for percutaneous coronary angioplasty or peripheral angioplasty in which an angioplasty balloon is used to dilate a lesion (e.g., calcified lesion) and restore normal blood flow in the artery. In this type of procedure, a catheter carrying a balloon is advanced into the vasculature along a guide wire until the balloon is aligned with calcified plaques. The balloon is then pressurized to reduce or break the calcified plaques and push them back into the vessel wall.

More recently, the assignee herein has developed a treatment system that includes electrodes within an angioplasty type balloon. In use, the balloon is advanced to the region of an occlusion. The balloon is then initially pressurized with a conductive fluid. A series of high voltage pulses are applied to the electrodes within the balloon, with each pulse generating a shock wave in the conductive fluid. The shock waves pass through the balloon wall and into the occlusion, cracking the calcified plaques. Once the calcified plaques are cracked, the balloon can be further expanded to open the vessel. Such system is disclosed in U.S. Pat. Nos. 8,956,371, 8,888,788, and U.S. Pub. No. 2019/0150960, all of which are incorporated herein by reference. Further, the assignee herein has developed techniques for providing an electrode on the tip of a guide wire for generating forward directed shock waves. This approach is disclosed in U.S. Patent Publication No. 2015/0320432, also incorporated herein by reference.

The present invention relates techniques for treating tight, hard-to-cross calcific lesions by positioning low-profile shock wave electrodes at the distal portion of the balloon, including the distal leg segment and/or the distal cone segment of the angioplasty balloon.

### BRIEF SUMMARY

The invention provides a device for generating shock waves. An exemplary device for generating shock waves comprises an elongated tube; a balloon wrapped circumferentially around a portion of the elongated tube, the balloon comprising: a distal end sealed to the elongated tube, a leg segment proximal to the distal end of the balloon, a cone segment proximal to the leg segment of the balloon, and a straight segment proximal to the cone segment of the balloon, wherein, when the balloon is inflated: the leg segment is substantially cylindrical or is of a tapered shape with an average diameter, the cone segment is of a tapered shape with an average diameter greater than the average diameter of the leg segment, and the straight segment is of a substantially cylindrical shape with a diameter greater than the average diameter of the cone segment; and at least one distal emitter configured to generate shock waves, wherein the at least one distal emitter is positioned in the leg segment or the cone segment of the balloon.

In some embodiments, at least one distal emitter is positioned in the leg segment of the balloon.

In some embodiments, at least one distal emitter is positioned in the cone segment of the balloon.

In some embodiment, the at least one distal emitter comprises a first distal emitter positioned in the leg segment of the balloon and a second distal emitter positioned in the cone segment of the balloon.

In some embodiments, the at least one distal emitter is located between approximately 0.5 mm and approximately 1 mm from a distal end of a gap between the leg segment of the balloon and the elongated tube.

In some embodiments, when the balloon is inflated, a gap between an inner diameter of the leg segment and an outer diameter of the elongated tube is between around 0.001″ to around 0.003″.

In some embodiments, the leg segment is less than approximately 3.5 mm long, and the cone segment is approximately 4.5 mm to approximately 5.5 mm long.

In some embodiments, when the balloon is inflated, a first vertex angle between the leg segment of the balloon and the elongated tube is smaller than 5 degrees.

In some embodiments, when the balloon is inflated, a second vertex angle between the cone segment of the balloon and the elongated tube is larger than the first vertex angle.

In some embodiments, the at least one distal emitter comprises a first electrode pair, the first electrode pair comprising: a conductive portion of a first insulated wire and a conductive portion of a second insulated wire.

In some embodiments, the conductive portion of the first insulated wire is formed by removing a first portion of insulation from the first insulated wire, and the conductive portion of the second insulated wire is formed by removing a second portion of insulation from the second insulated wire.

In some embodiments, the first insulated wire comprises a flattened distal segment, and the first portion of insulation is removed from the flattened distal segment of the first insulated wire, and the second insulated wire comprises a flattened distal segment, and the second portion of insulation is removed from the flattened distal segment of the second insulated wire.

In some embodiments, a diameter of the flattened distal segment of the first insulated wire is approximately 47% to 75% of a diameter of a proximal segment of the first insulated wire, and a diameter of the flattened distal segment of the second insulated wire is approximately 47% to 75% of a diameter of a proximal segment of the second insulated wire.

In some embodiments, a layer of polymer covers at least a portion of the first insulated wire and the second insulated wire, such that the conductive portion of the first insulated wire is held a controlled distance apart from the conductive portion of the second insulated wire.

In some embodiments, the at least one distal emitter further comprises a second electrode pair, the second electrode pair comprising: a further conductive portion of the second insulated wire and a conductive portion of a third insulated wire.

In some embodiments, the at least one distal emitter further comprises a second electrode pair and a third electrode pair, wherein the second electrode pair comprises: a further conductive portion of the second insulated wire and a first side edge of a conductive sheath wrapped circumferentially around the elongated tube; and wherein the third electrode pair comprises: a second side edge of the conductive sheath and a conductive portion of a third insulated wire.

In some embodiments, the at least one distal emitter comprises a first electrode pair and a second electrode pair, wherein the first electrode pair comprises: a conductive portion of a first insulated wire and a first side edge of a conductive sheath wrapped circumferentially around the elongated tube; and wherein the second electrode pair comprises: a second side edge of the conductive sheath and a conductive portion of a second insulated wire.

In some embodiments, the first side edge and the second side edge are positioned circumferentially 180 degrees on opposite edges of the conductive sheath.

In some embodiments, the elongated tube is tapered toward the distal end.

In some embodiments, the device comprises at least one proximal emitter configured to generate shock waves, wherein the at least one proximal emitter is positioned in the straight segment of the balloon.

In some embodiments, the device comprises a variable high voltage pulse generator selectively connected to the at least one distal emitter and the at least one proximal emitter, wherein the variable high voltage pulse generator can be activated to generate shock waves at either the at least one distal emitter or the at least one proximal emitter.

An exemplary method of generating shock waves comprises introducing a shock wave device into a patient's vasculature, the shock wave device comprising: a balloon comprising: a distal end, a leg segment proximal to the distal end of the balloon, a cone segment proximal to the leg segment of the balloon, and a straight segment proximal to the cone segment of the balloon; at least one distal emitter in the leg segment or the cone segment of the balloon; and at least one proximal emitter in the straight segment of the balloon. During a first stage, the method includes advancing the shock wave device in the patient's vasculature such that the distal leg segment of the balloon is advanced as far into the calcified lesion as possible; inflating the balloon with a conductive fluid such that the inflated balloon is gently fixed to walls of the vasculature in direct proximity with the calcified lesion, wherein, when the balloon is inflated, the leg segment is substantially cylindrical or is of a tapered shape with an average diameter, the cone segment is of a tapered shape with an average diameter greater than the average diameter of the leg segment, and the straight segment is of a substantially cylindrical shape with a diameter greater than the average diameter of the cone segment; activating a voltage source to generate shock waves at the at least one distal emitter; and after activating the voltage source, deflating the balloon. During a second stage, the method includes further advancing the shock wave device in the patient's vasculature such that the straight segment of the balloon is advanced as far into the calcified lesion as possible; inflating the balloon with the conductive fluid such that the inflated balloon is gently fixed to walls of the vasculature in direct proximity with the calcified lesion; and activating the voltage source to generate shock waves at the at least one proximal emitter.

In some embodiments, the first stage is repeated until the calcified lesion is modified such that the straight segment of the balloon is able to advance into the calcified lesion.

In some embodiments, activating the voltage source during the second stage generates shock waves at both the at least one proximal emitter and the at least one distal emitter.

In some embodiments, the at least one distal emitter is positioned in the leg segment of the balloon

In some embodiments, the at least one distal emitter is positioned in the cone segment of the balloon.

In some embodiments, the at least one distal emitter comprises a first distal emitter positioned in the leg segment of the balloon and a second distal emitter positioned in the cone segment of the balloon.

The invention also provides a device for generating shock waves. An exemplary device for generating shock waves comprises a balloon comprising: a distal end, a leg segment proximal to the distal end of the balloon, a cone segment proximal to the leg segment of the balloon, wherein, when the balloon is inflated: the leg segment is cylindrical or is of a tapered shape with a first vertex angle, and the cone segment is of a tapered shape with a second vertex angle larger than the first vertex angle, an elongated tube extending through the balloon, wherein the balloon wraps around a longitudinal segment of the elongated tube, wherein the distal end of the balloon is sealed to the elongated tube; an electrode pair between the elongated tube and the leg segment of the balloon, comprising: a conductive portion of a first insulated wire extending over the elongated tube, a conductive portion of a second insulated wire extending over the elongated tube, wherein, when a voltage is applied across the first insulated wire and the second insulated wire, a current is configured to flow from the first insulated wire to the second insulated wire, and wherein a shock wave is created across the conductive portion of the first insulated wire and the conductive portion of the second insulated wire.

In some embodiments, the electrode pair is a first electrode pair, and wherein the device comprises a second electrode pair within the cone segment of the balloon.

In some embodiments, the second electrode pair comprises: a conductive portion of the second insulated wire extending over the elongated tube; and a conductive portion of a third insulated wire extending over the elongated tube.

In some embodiments, the second electrode pair comprises: a conductive portion of the second insulated wire extending over the elongated tube; and a conductive sheath circumferentially mounted around the elongated tube.

In some embodiments, the balloon further comprises a straight segment proximal to the cone segment; the devices further comprises a third electrode pair between the elongated tube and the straight segment of the balloon.

In some embodiments, the first electrode pair and the second electrode pair are configured to be driven by a first voltage source, and the third electrode pair is configured to be driven by a second voltage source different from the first voltage source.

In some embodiments, the conductive portion of the first insulated wire is formed by removing a first portion of insulation from the first insulated wire, and wherein the conductive portion of the second insulated wire is formed by removing a second portion of insulation from the second insulated wire.

In some embodiments, the first insulated wire comprises a flattened distal segment, and the first portion of insulation is removed from the flattened distal segment of the first wire, and the second insulated wire comprises a flattened distal segment, and the second portion of insulation is removed from the flattened distal segment of the second wire.

In some embodiments, the distal segment of the first insulated wire and the distal segment of the second insulated wire are secured to the elongated tube via wrapping material.

In some embodiments, the first vertex angle is smaller than 5 degrees.

In some embodiments, wherein the second vertex angle is larger than 5 degrees.

In some embodiments, the elongated tube comprises longitudinal grooves that are spaced apart by 90 degrees, and wherein a first portion of the first wire is placed in a first longitudinal groove and a second portion of the first wire is placed in a second longitudinal groove that is 90 degree from the first groove.

An exemplary method of generating shock waves comprises introducing a shock wave device into a patient's vasculature, the shock wave device comprising: a balloon comprising: a distal end, a leg segment proximal to the distal end of the balloon, a cone segment proximal to the leg segment of the balloon, and a straight segment proximal to the cone segment of the balloon; a first plurality of electrode pairs in the leg segment or the cone segment of the balloon; and a second plurality of electrode pairs in the straight segment of the balloon; advancing the shock wave device within the vasculature such that the leg segment of the balloon is at a treatment site; inflating the balloon with a conductive fluid, wherein, when the balloon is inflated, the leg segment is cylindrical or is of a tapered shape with a first vertex angle, the cone segment is of a tapered shape with a second vertex angle larger than the first vertex angle, the straight segment is of a cylindrical shape; and activating a first voltage source, wherein the first voltage source is configured to generate shock waves at the first set of electrode pairs but not the second set of electrode pairs.

In some embodiments, the method further comprises: after activating the first voltage source, deflating the balloon; advancing the shock wave device; inflating the balloon with a conductive fluid; activating a first voltage source and a second voltage source simultaneously, wherein the second voltage source is configured to generate shock waves at the second set of electrode pairs.

In some embodiments, the method further comprises: after activating the first voltage source, deflating the balloon; advancing the shock wave device; inflating the balloon with a conductive fluid; activating a second voltage source, wherein the second voltage source is configured to generate shock waves at the second set of electrode pairs.

### DESCRIPTION OF THE FIGURES

FIG. 1A depicts a prior art shock wave angioplasty device having a plurality of electrode assemblies.

FIG. 1B depicts an exemplary shock wave angioplasty device having a plurality of electrode assemblies, in accordance with some embodiments.

FIG. 1C depicts a set of shock wave electrode assemblies in an exemplary shock wave angioplasty device that may be activated to generate shock waves at multiple locations, in accordance with some embodiments.

FIG. 1D depicts the connectivity between a plurality of inner electrodes and sheaths to attain the configuration of FIG. 1B, in accordance with some embodiments.

FIG. 1E schematically depicts an electrical diagram of the configuration of FIG. 1B, in accordance with some embodiments.

FIG. 1F schematically depicts an electrical diagram of the configuration of FIG. 1B, in accordance with some embodiments.

FIG. 2A depicts a set of shock wave electrode assemblies in an exemplary shock wave angioplasty device that may be activated to generate shock waves at multiple locations, in accordance with some embodiments.

FIG. 2B depicts the connectivity between a plurality of inner electrodes and sheaths to attain the configuration of FIG. 2A, in accordance with some embodiments.

FIG. 2C schematically depicts an electrical diagram of the configuration of FIG. 2A, in accordance with some embodiments.

FIG. 2D schematically depicts an electrical diagram of the configuration of FIG. 2A, in accordance with some embodiments.

FIG. 3A depicts a set of shock wave electrode assemblies in an exemplary shock wave angioplasty device that may be activated to generate shock waves at multiple locations, in accordance with some embodiments.

FIG. 3B depicts the connectivity between a plurality of inner electrodes and sheaths to attain the configuration of FIG. 3A, in accordance with some embodiments.

FIG. 3C schematically depicts an electrical diagram of the configuration of FIG. 3A, in accordance with some embodiments.

FIG. 3D schematically depicts an electrical diagram of the configuration of FIG. 3A, in accordance with some embodiments.

FIG. 4 depicts an exemplary shock wave electrode assembly comprising a conductive sheath and two wires, in accordance with some embodiments.

FIG. 5 depicts another exemplary set of shock wave electrode assemblies, in accordance with some embodiments.

FIG. 6 depicts another exemplary set of shock wave electrode assemblies, in accordance with some embodiments.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

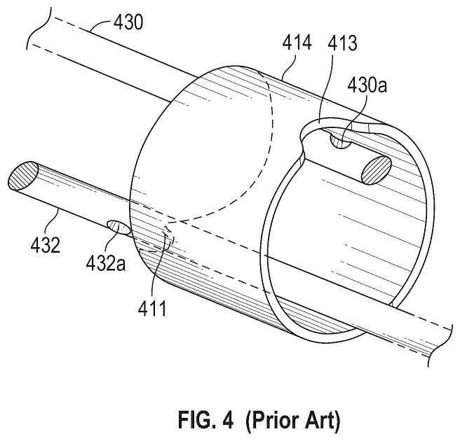

The assignee herein has developed a number of low-profile shock wave electrodes that may be suitable for use in angioplasty and/or valvuloplasty procedures. For example, in U.S. Pub. No. 2019/0150960, the assignee discloses a low-profile electrode assembly, in which an outer electrode is formed by a conductive sheath, and an inner electrode is formed by removing a portion of an insulated wire (e.g., cutting a hole in the insulating layer near the end of the wire) to expose an electrically conductive portion of the insulated wire. The inner electrode is placed a controlled distance apart from the side edge of the conductive sheath (e.g., as shown in FIG. 4) to allow for a reproducible arc for a given current and voltage.

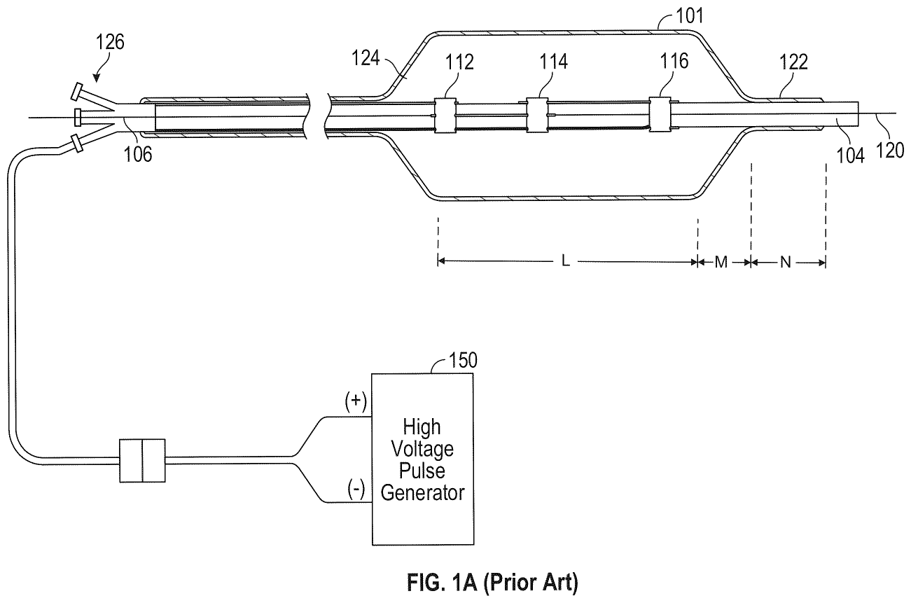

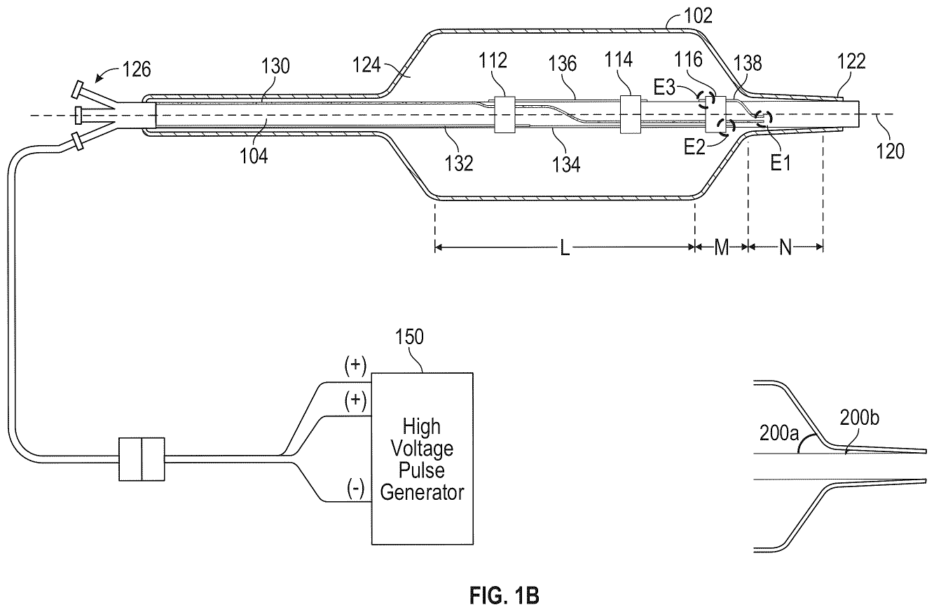

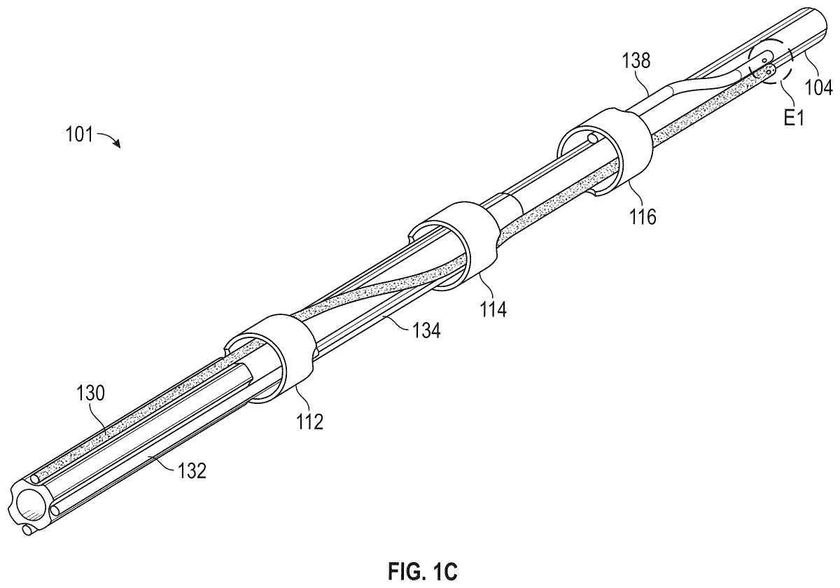

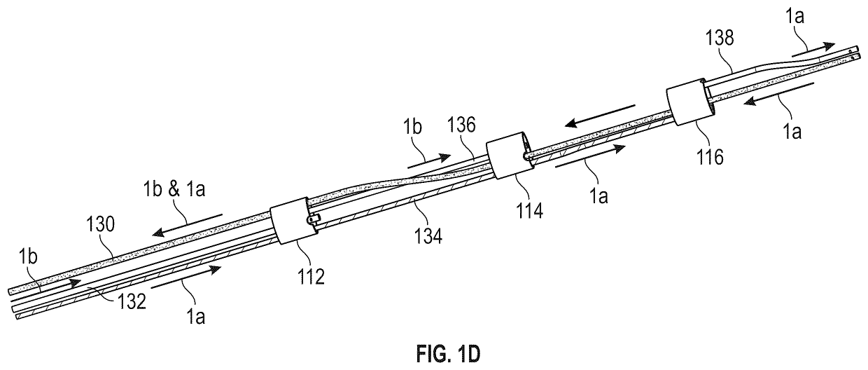

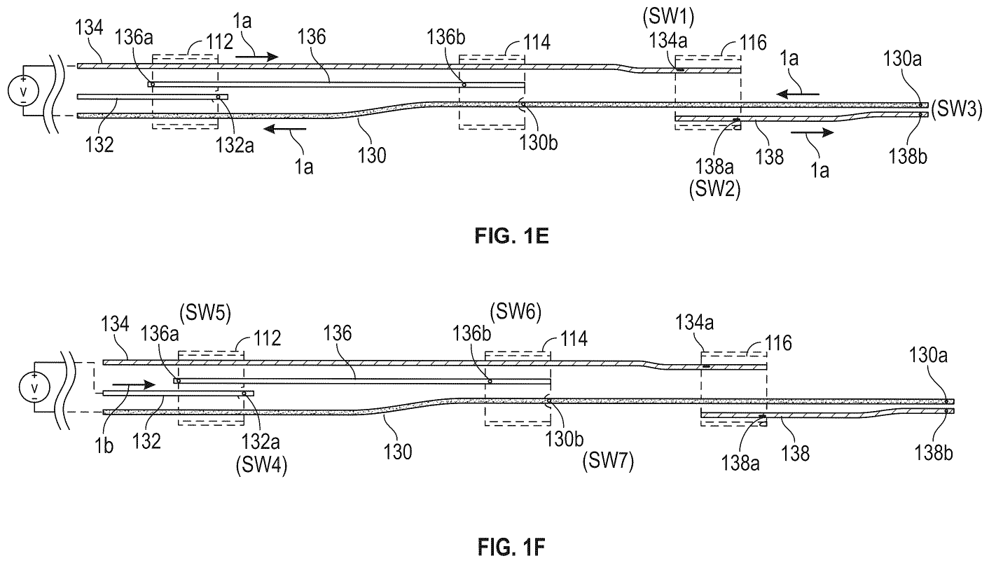

FIG. 1A depicts a prior art shock wave angioplasty device according to assignee's prior filing U.S. Pub. No. 2019/0150960. The shock wave device includes an elongated tube 104 and an angioplasty balloon 101. The angioplasty balloon wraps circumferentially around a portion of the elongated tube 104 in a sealed configuration via, for example, a seal 122. The angioplasty balloon 101 forms an annular channel 124 around the elongated tube 104 through which a conductive fluid, such as saline, may be admitted into the balloon via fill ports 126. The balloon is filled with the fluid such that the balloon can be inflated and gently fixed to the walls of the artery in direct proximity with a calcified lesion. In some embodiments, the fluid may also contain an x-ray contrast to permit fluoroscopic viewing of the catheter during use.

The elongated tube 104 includes a number of longitudinal grooves or channels configured for retaining wires and/or inner electrodes. A plurality of insulated wires are placed within the grooves of the elongated tube 104. Furthermore, a number of conductive sheaths 112, 114, and 116 are circumferentially mounted around the elongated tube 104. A variable high voltage pulse generator 150 is connected to two of the insulated wires. The insulated wires and the sheaths form three electrode assemblies that can be activated to generate shock waves at 6 locations (e.g., along the length of the vessel), as discussed in U.S. Pub. No. 2019/0150960.

The prior art system in FIG. 1A can have a lower crossing profile than contemporary intravascular lithotripsy (“IVL”) catheters. However, when the prior art system is used to treat tighter calcified lesions (e.g., having a crossing area smaller than the crossing profile of the prior art system), the prior art system still has drawbacks. As depicted in FIG. 1A, the sheaths 112, 114, and 116 are positioned within the straight segment (labelled “L” in FIG. 1A) of the angioplasty balloon 101, thus generating shock waves only within the straight segment of the balloon. As a result, during treatment, a physician has to advance the balloon catheter until the straight segment (labelled “L”) is positioned within the treatment site prior to delivering shock waves. If the calcified lesion is difficult to cross for the prior art system given the cross profile of the straight segment of the balloon (which can be larger than the cross profile of the cone segment and the leg segment due to the size of the conductive sheath and/or the amount of balloon material), additional pre-dilatation and/or pre-treatment devices are needed to ensure optimal positioning of the balloon. The use of a pre-dilatation and/or pre-treatment device adds cost and complexity to the procedure.

Described herein are IVL catheters with one or more miniaturized lithotripsy emitters positioned at the distal segment of the angioplasty balloon (i.e., the distal leg segment “N” and/or distal cone segment “M” of the balloon). Positioning the emitters at the distal portion of the balloon allows IVL treatment in tighter, hard-to-cross calcific lesions and facilitates further crossing of the IVL balloons. The present invention is similar to the prior art system in FIG. 1A in that it can comprise an array of lithotripsy emitters within the straight section of the balloon (labelled “L” in FIG. 1A) to deliver shock waves. In addition, the present invention can further comprise low-profile emitters inside the cone segment (labelled “M”) and distal leg segment (labelled “N”) to facilitate IVL treatment in tight, hard-to-cross calcific lesions.

FIG. 1B depicts an exemplary shock wave angioplasty device 100 according to an embodiment of the invention. The shock wave device 100 includes an elongated tube 104 and an angioplasty balloon 102. The angioplasty balloon wraps circumferentially around a portion of the elongated tube 104 in a sealed configuration via, for example, a seal 122. The angioplasty balloon 102 forms an annular channel 124 around the elongated tube 104 through which a conductive fluid, such as saline, may be admitted into the balloon via fill ports 126. The balloon is filled with the fluid such that the balloon can be inflated and gently fixed to the walls of the artery in direct proximity with a calcified lesion. In some embodiments, the fluid may also contain an x-ray contrast to permit fluoroscopic viewing of the catheter during use.

FIG. 1B depicts the angioplasty balloon 102 in an inflated state. As depicted, the balloon includes three longitudinal segments at the distal end: a straight segment (labelled “L”), a cone segment (labelled “M”), and a leg segment (labelled “N”). When inflated, the straight segment is configured to expand into a cylindrical shape or substantially cylindrical shape; the cone segment is configured to expand into a cone or tapered shape; the leg segment is configured to provide no or little expansion. Each of the longitudinal segments has a diameter, which is equal to the distance between a point on the surface of the segment to a corresponding point 180 degrees circumferentially across the balloon. Each of the segments additionally has an average diameter, which is approximated as the sum of the maximum diameter of the segment and the minimum diameter of the segment divided by two. As depicted in FIG. 1B, when inflated, the cone segment has an average diameter that is greater than the average diameter of the leg segment. The straight segment has a diameter greater than the average diameter of the cone segment. As shown in FIG. 1B, when the balloon is inflated, the leg segment is of a cone shape. The vertex angle of the cone segment (200a) is larger than the vertex angle of the leg segment (200b). In some embodiments, the vertex angle 200a is between 5-10° and the vertex angle 200b is between 0-5°. In some embodiments, the vertex angle 200a is between 5-20° and the vertex angle 200b is between 0-5°. In some embodiments, when the balloon is inflated, the leg segment is of a cylindrical shape (i.e., 200b is 0 degree).

By design, there is a gap between the inner diameter of the leg segment of the balloon and the outer diameter of the elongated tube 104 to accommodate a distal emitter (labelled “E1”) formed by the distal portions of two wires. Thus, when the balloon is inflated with fluid, the gap is filled with the fluid to allow generation of shock waves at the distal emitter (labelled “E1”). In some embodiments, the gap is between around 0.001″-0.003″. In general, the bigger gap (thus more fluid) between the emitter and the balloon wall, the less likely pulsing can negatively impact the balloon integrity. In some embodiments, E1 can be located at ˜0.5-1 mm of the distal end of the gap. In some embodiments, the leg segment is approximately 3.5-5.0 mm long, and the cone segment is approximately 4.5-5.5 mm long.

Based on design, the leg segment in some embodiments provides no or little expansion when the balloon is inflated. In a deflated state, the leg segment can be of cylindrical, substantially cylindrical, or slightly tapered (vertex angle smaller than 5 degrees). In an inflated state, the leg segment can be of cylindrical, substantially cylindrical, or slightly tapered (vertex angle smaller than 5 degrees).

The elongated tube 104 includes a number of longitudinal grooves or channels configured for retaining wires and/or inner electrodes. The elongated tube 104 also includes a lumen through which a guide wire 120 is inserted. In the depicted example in FIG. 1B, insulated wires 130, 132, 134, 136, and 138 are placed along the elongated tube 104 (e.g., within the grooves of the elongated tube 104). Furthermore, a number of conductive sheaths 112, 114, and 116 are circumferentially mounted around the elongated tube 104. A variable high voltage pulse generator 150 can provide a first voltage channel between wire 130 and 134 and a second voltage channel between 130 and 132. The insulated wires and the sheaths form seven electrode pairs that can be activated to generate shock waves at 7 locations (e.g., along the length of the vessel) simultaneously or in multiple stages of the IVL treatment, as discussed in detail below.

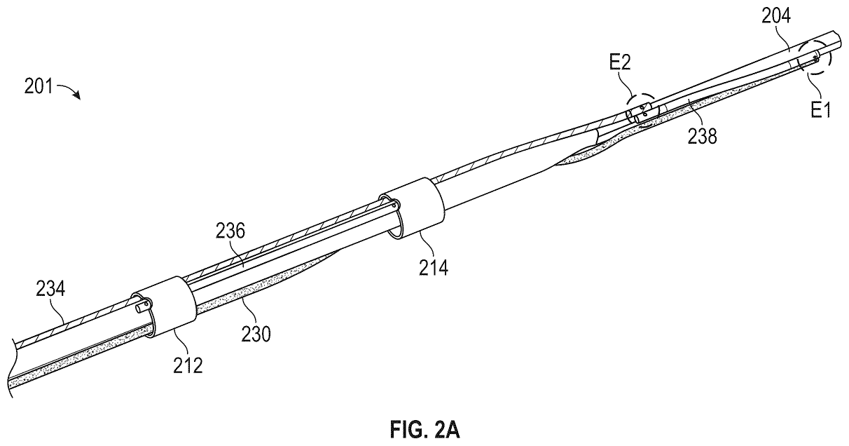

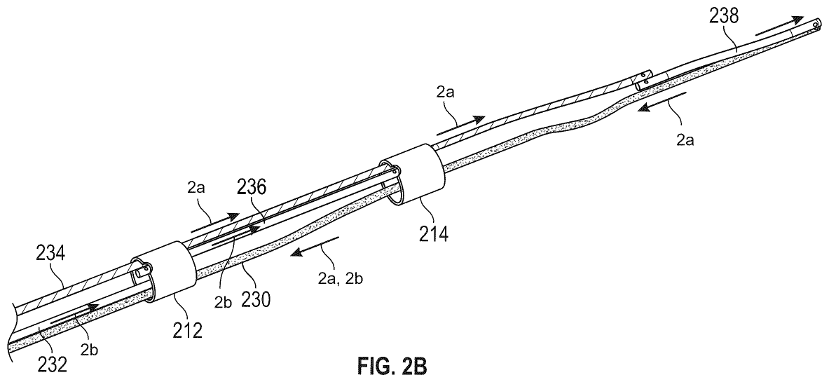

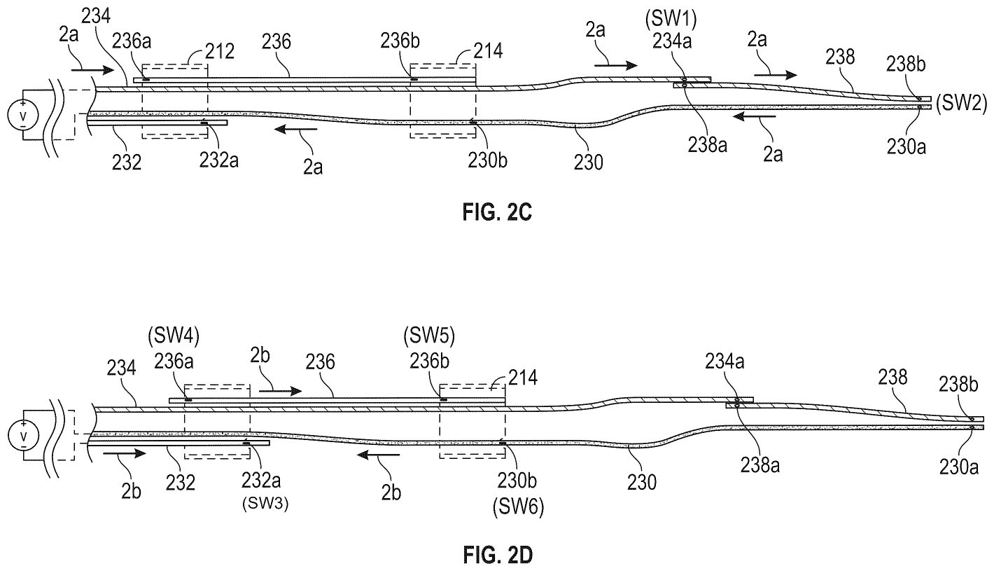

As shown in FIG. 1B, the second most distal emitter (labelled “E2”) and the third most distal emitter (labelled “E3”) are placed in the cone segment of the balloon. In the depicted example, E2 is formed by the distal side edge of the sheath 116 and a distal portion of the wire 134. In other embodiments (e.g., FIGS. 2A), E2 can be formed by the distal portions of two wires similar to E1.

Accordingly, the exemplary shock wave angioplasty device 100 can generate shock waves in the distal segments (“M” and “N”) in addition to the straight segment (“L”) of the angioplasty balloon. This is advantageous because the distal segments have a lower profile than the straight segment when the balloon is deflated (e.g., due to the lower amount of surrounding balloon material in the distal segments, due to the use of low-profile wire-to-wire emitters in the distal segments, due to the tapering design of the cone and the leg) and when the balloon is inflated. This can facilitate IVL treatment in tight, hard-to-cross calcific lesions.

In operation, a physician advances the guide wire 120 from an entry site on a patient (e.g., an artery in the groin area of the leg) to the target region of a vessel (e.g., a region having calcified plaques that need to be broken up). The shock wave device shown in FIG. 1B, which comprises the elongated tube 104 with a guide wire lumen and the angioplasty balloon may be advanced over the guide wire to the target region of the vessel. The balloon may be collapsed over the elongated member while the device is advanced through the vasculature. The location of the shock wave device may be determined by x-ray imaging and/or fluoroscopy.

The balloon distal leg segment and cone segment are advanced as far as possible inside the tight lesion. A guide catheter is positioned such that its distal tip is proximal to sheath 116 to retain the deflated state of the straight portion of the balloon. The balloon is then inflated by a conductive fluid (e.g., saline and/or saline mixed with an image contrast agent) to IVL pressure, allowing conductive fluid to fill the leg segment and cone segment of the balloon. In the first stage of the IVL treatment, a voltage is applied between wires 130 and 134 to activate the three most distal emitters E1, E2, and E3 (e.g., for 1 cycle). After the completion of the 1^{st} cycle, the balloon is deflated and the balloon distal leg and cone segments are advanced further into the lesion. The balloon is again inflated and another cycle of therapy is applied. Further advancement of the balloon is attempted after the completion of the cycle. In some embodiments, the other emitters are not activated during the first stage of the treatment. In some embodiments, the straight portion of the balloon is retained by a guide catheter until the straight portion of the balloon is able to advance and cross the lesion. After calcium in the tight lesion has been modified and the straight portion of the balloon is able to cross the lesion, the balloon is again inflated to IVL pressure.

In the second stage of the IVL treatment, all of the emitters are activated simultaneously by applying a first voltage across wire 130 and 134 and a second voltage across 130 and 132. In some embodiments, the 3 distal emitters (i.e., E1, E2, and E3) may not be activated if the straight portion of the balloon is able to cross the entire lesion. For example, the physician may simultaneously connect the insulated wire 134 to a first positive lead of the pulse generator, connect the insulated wire 132 to a second positive lead of the pulse generator, and connect the insulated wire 130 to a negative lead or the ground. Alternatively, the physician may only connect the insulated wire 132 to a positive lead of the pulse generator and connect the insulated wire 130 to the negative lead or ground. Shock waves generated by the proximal emitters (i.e., the emitters proximal to the three distal emitters) provide additional treatment to the calcific lesion, while shock waves generated by the three distal emitters E1-E3 allows further advancement of the balloon. The magnitude of the shock waves can be controlled by controlling the magnitude of the pulsed voltage, the current, the duration, and the repetition rate. The physician may start with low energy shock waves and increase the energy as needed to crack the calcified plaques. Such shock waves will be conducted through the fluid, through the balloon, through the blood and vessel wall to the calcified lesion where the energy will break the hardened plaque. The progress of the plaque break-up may be monitored by x-ray and/or fluoroscopy. Once the calcified lesion has been sufficiently treated, the balloon may be inflated further, then deflated, and the shock wave device and guide wire may be withdrawn from the patient.

As such, embodiments of the present invention amount to a crossing and IVL combination catheter. Emitters in the distal balloon leg and cone segments are used to facilitate crossing of the balloon catheter in tight, hard-to-cross lesions. Emitters within the straight segment deliver IVL treatment and dilatation to calcified lesions. In some embodiments, the angioplasty balloon is made of soft grade polymer extrusion (e.g., certain Pebax material) allowing the distal cone and leg segments to withstand higher heat and pressure from IVL treatment.

FIG. 1C depicts exemplary shock wave electrode assemblies included in the exemplary shock wave angioplasty device 100 of FIG. 1B, according to some embodiments of the invention. The elongated tube 104 provides four longitudinal grooves, as indicated by the proximal end of the elongated tube. In some embodiments, the elongated tube 104 is tapered toward its distal end. A number of insulated wires (e.g., lead or interstation wires) 130, 132, 134, and 136 are disposed on the outer surface of the elongated tube 104 such that they extend along the length of the elongated tube. The wires can be straight or twisted. For example, wire 132 is disposed within one groove of the elongated tube 104, while wire 130 is twisted and different segments of the wire 130 are disposed along different grooves or outer surface of the elongated tube 104.

The shock wave angioplasty device 100 further includes three conductive sheaths 112, 114, and 116 each circumferentially mounted around the elongated tube 104. FIG. 1D depicts the relative placement of the wires and the conductive sheaths, with the elongated tube removed. FIG. 1D further depicts two current flows 1a and 1b for generating seven sources of shock waves, as described with reference to FIGS. 1E and 1F.

An emitter can be formed by a side edge of a conductive sheath and a portion of a wire, as described in assignee's prior filing U.S. Pub. No. 2019/0150960. For example, as shown in FIG. 4, a portion of the insulating layer of the wire 430 is removed near the distal end of the wire 430 to expose an electrically conductive wire portion, forming the first inner electrode 430a. In the depicted example, a hole in the insulating layer is cut on the curved outer surface along the length of the wire. The removed portion may be in any shape, such as a circle, a rectangle, a strip around the circumference of the wire, etc. The location, shape, and size of the removed portion may vary to control the location, direction, and/or magnitude of the shock wave. In some embodiments, an inner electrode may be formed by cutting the end of the wire to expose an electrically conductive cross-section of the wire. In some embodiments, flat wires rather than round wires are used to further reduce the crossing profile of the electrode assembly.

As shown in FIG. 4, the first inner electrode 430a is adjacent to, but not in contact with, a distal side edge 413 of the conductive sheath 412. The conductive sheath 412 functions as an outer electrode, and the inner electrode 430a is placed a controlled distance apart from the distal side edge 413 of the first conductive sheath to allow for a reproducible arc for a given voltage and current. The electrical arcs are then used to generate shock waves in the conductive fluid. In operation, a first shock wave is created across the inner electrode 430a and the distal side edge 413 of the conductive sheath 412. In a similar manner, a portion of the insulated wire 432 is removed to form a second inner electrode 432a. Specifically, a portion of the insulating layer of the wire 432 is removed near the proximal end of the wire 432 to expose an electrically conductive wire portion along the length of the wire, forming the second inner electrode 432a. As shown, the second inner electrode 432a is adjacent to, but not in contact with, a proximal side edge 411 of the conductive sheath 412. Further, the first inner electrode 430a and the second inner electrode 432a are positioned circumferentially 180 degrees from each other. In operation, the conductive sheath 412 acts as an outer electrode and a second shock wave is created across the second inner electrode 432a and the proximal side edge 411 of the first conductive sheath 412. The diagonal placement of the inner electrodes allows the sonic output to be distributed more evenly longitudinally along the balloon while making the shock waves less annular. One of ordinary skill in the art should recognize that the location of a shock wave can be configured in a flexible manner by arranging the corresponding wire and the corresponding conductive sheath (and the location of the corresponding cut-out on the sheath, if available) accordingly.

As described in assignee's prior filing U.S. Pub. No. 2019/0150960, two emitters can be formed along the same side edge of the sheath; further, a shock wave can be generated between an inner electrode and a straight side edge of the conductive sheath that does not include any cut-outs.

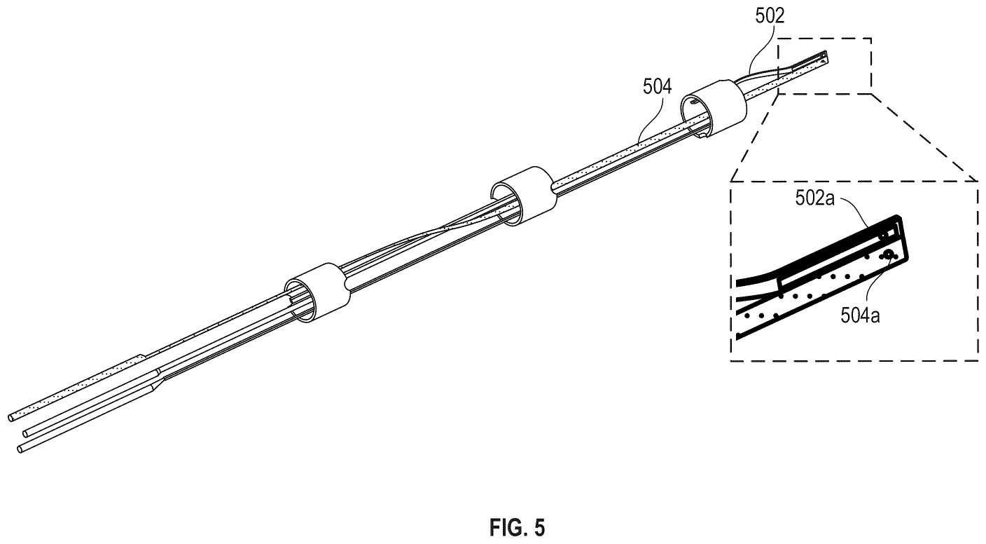

An emitter can also be formed by the distal portions of two wires, as depicted in FIG. 5. A shown, a portion of the insulating layer of the wire 502 is removed near the distal end of the wire to expose an electrically conductive wire portion, forming an electrode 502a. Further, a portion of the insulating layer of the wire 504 is removed near the distal end of the wire to expose an electrically conductive wire portion, forming an electrode 504a. The pair of electrodes are adjacent to, but not in contact with, each other. Specifically, the pair of electrodes are placed a controlled distance apart to allow for a reproducible arc for a given voltage and current. The electrical arcs are then used to generate shock waves in the conductive fluid.

The wires 502 and 504 can be flattened to reduce the profile of the electrode pair. As shown in FIG. 5, while the proximal end of the wire 504 retains its original shape (i.e., cylindrical), the distal segment of the wire 504 has been flattened to reduce its profile. In some embodiments, a wire of 0.0053″-0.0064″ in diameter can be flattened to 0.0030″-0.0040″ in diameter, resulting in the flattened distal segment of the wire having a diameter that is approximately 47% to 75% relative to the diameter of the unflattened proximal segment of the wire. Having wires in the distal leg may affect the dimension and flexibility of the device, thus deliverability.

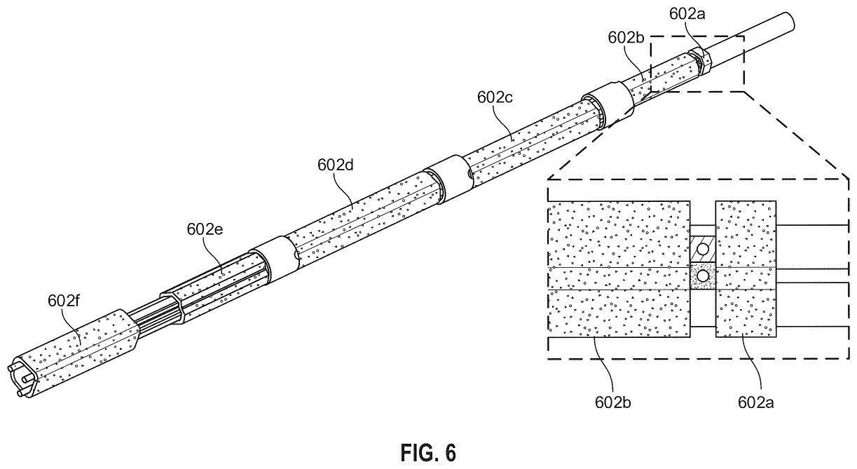

In some embodiments, a layer of polyethylene terephthalate (“PET”) can cover the wires before and after the cutouts to hold down the wires. As shown in FIG. 6, a plurality of PET bands 602a-f are used to secure the wires to the elongated tube. Specifically, PET bands 602a and 602b covers the two wires forming the distal electrode pair before and after the cutouts. In some embodiments, these cutouts are recessed from the top surface by 0.00025″ (thickness of the PET).

FIGS. 1E and 1F schematically depict two electrical diagrams of the configuration of FIGS. 1B-D, in accordance with some embodiments. With reference to FIG. 1E, when a first high voltage is applied (e.g., using the high voltage pulse generator 150 of FIG. 1B) across the proximal end of the insulated wire 130 and the proximal end of the insulated wire 134, a current 1a may flow as indicated by the arrows, with the insulated wire 130 as the common ground wire (i.e., connecting to a ground or negative channel). As shown, the current flows from the proximal end of the insulated wire 134 toward to the distal end of the insulated wire 134 and, via the insulation removed spot that is electrically conductive (i.e., inner electrode 134a), to the proximal side edge of the conductive sheath 116 (i.e., outer electrode). The duration and the magnitude of the voltage pulse are set to be sufficient to generate a gas bubble at the surface of the inner electrode 134a a causing a plasma arc of electric current to traverse the bubble and create a rapidly expanding and collapsing bubble, which creates the mechanical shock wave in the balloon (“SW1”). The size of the bubble and the rate of expansion and collapse of the bubble (and therefore the magnitude, duration, and distribution of the mechanical force) may vary based on the magnitude and duration of the voltage pulse, as well as the distance between the inner and outer electrodes, the surface area of the electrodes, and/or the shape of the outer electrode (e.g., whether there is an arcuate cut-out on the side edge).

The current 1a may further traverse from the distal side edge of the conductive sheath 116 (i.e., outer electrode) to the insulated wire 138, via the insulation removed spot near the proximal end of the insulated wire 138 (i.e., inner electrode 138a). The voltage pulse may create a potential difference high enough to form a plasma arc between them, generating a bubble that gives rise to a second shock wave (“SW2”). In the depicted example, the inner electrode 134a and the inner electrode 138a a are located circumferentially opposite to each other (e.g., 180 degrees apart around the circumference of the elongated tube), and thus the first shock wave and the second shock wave may propagate in opposite directions, extending outward from the side of the elongated tube.

The current 1a may further traverse to the distal end of the wire 138 and, via the insulation removed spot that is electrically conductive near the distal end of the wire 138 (i.e., the electrode 138b), to the insulation removed spot that is electrically conductive near the distal end of the wire 130 (i.e., the electrode 130a). The high voltage pulse generator may apply a voltage pulse such that the potential difference between the 138b and 130a is high enough to form a plasma arc between them, generating a bubble that gives rise to a third shock wave (“SW3”). Similar to the 1^{st} and 2^{nd} shockwaves, the shockwave propagates and extends outward from the 2 insulation removed spots 138b and 130a.

The current then returns to the voltage source generator via the insulated wire 130 to a voltage output port (not depicted), which may be a negative channel or a ground channel. Optionally, a connector (not depicted) may be provided between the insulated wires 134 and 130 and the voltage pulse generator so that the wires may be easily connected to the output ports of the high voltage generator.

With reference to FIG. 1F, when a second high voltage is applied (e.g., using the high voltage pulse generator 150 of FIG. 1B) across the proximal end of the insulated wire 130 and the proximal end of the insulated wire 132, a current 1b may flow as indicated by the arrows, with the insulated wire 130 as the common ground wire (i.e., connecting to a ground or negative channel). As shown, the current flows from the proximal end of the insulated wire 132 toward to the distal end of the insulated wire 132 and, via the insulation removed spot that is electrically conductive (i.e., inner electrode 132a), to the distal side edge of the conductive sheath 112 (i.e., outer electrode), generating shock waves (“SW4”). The current 1b may further traverse from the proximal side edge of the conductive sheath 112 (i.e., outer electrode) to the insulated wire 136, via the insulation removed spot near the proximal end of the insulated wire 136 (i.e., inner electrode 136a), generating shock waves (“SW5”). As shown, 132a and 136a are positioned circumferentially 180 degrees from each other on opposite edges of the conductive sheath 112.

The current 1b may further traverse toward the distal end of the wire 136 and, via the insulation removed spot that is electrically conductive near the distal end of the wire 136 (i.e., the electrode 136b), to the proximal side edge of conductive sheath 114, generating shock waves (“SW6”). The current 1b may further traverse from the distal side edge of the conductive sheath 114) to the insulated wire 130, via the insulation removed spot on the insulated wire 130 (i.e., inner electrode 130b), generating shock waves (“SW7”). The current then returns to the voltage source generator via the insulated wire 130 to a voltage output port. As shown, 136b and 130b are positioned circumferentially 180 degrees from each other on opposite edges of conductive sheath 114.

The current flows depicted in FIGS. 1E and 1F can occur simultaneously (e.g., by applying two voltage channels simultaneously) or at different times depending on the stage of the treatment. In the exemplary treatment discussed above, only current 1a is activated during the first stage of the treatment, thus activating only SW1, SW2, and SW3. This is to break loose calcifications in a tight lesion that only the distal segments (e.g., distal cone and/or distal leg) of the balloon can cross or come close to. After calcium in the tight lesion has been modified, the balloon is deflated and advanced further into the lesion. The balloon is again inflated to IVL pressure. In the second stage of the IVL treatment, all of the emitters are activated simultaneously. Shock waves generated by the proximal emitters (i.e., SW4, SW5, SW6, and SW7) provide additional treatment to the calcific lesion, while shock waves generated by the three distal emitters (i.e., SW1, SW2, SW3) allows further advancement of the balloon. Alternatively, only SW4, SW5, SW6, and SW7 are activated in the 2^{nd} stage.

In some embodiments, a multiplexor may be used with the high voltage pulse generator to control application of pulses. This may allow shock waves with different frequency, magnitude, and timing to be generated along the elongated tube. In the depicted embodiment in FIG. 1E-F, the two voltage channels share the same common ground wire (i.e., insulated wire 130). One of ordinary skill in the art should understand that any number of voltage channels (e.g., 4) may be configured around a single elongated tube, and these voltage channels may rely on the same or different common ground wires.

FIGS. 2A-D depict another exemplary shock wave electrode assemblies 201 that can be included in the exemplary shock wave angioplasty device 100 of FIG. 1B, according to some embodiments of the invention. While the embodiment 101 in FIG. 1C includes only one wire-to-wire electrode pair (i.e., labelled “E1” in FIG. 1C), the embodiment 201 includes two wire-to-wire electrode pairs (labelled “E1” and “E2” in FIG. 2A). In some embodiments, both E1 and E2 are disposed within the leg segment of the balloon; in some embodiments, E1 is disposed within the leg segment while E2 is disposed within the cone segment of the balloon.

Comparing to the embodiment 101 in FIG. 1C, the embodiment 201 has a longer distal segment that is extremely low-profile (i.e., profile smaller than the cross section of a conductive sheath). Thus, when treating a calcified lesion that has a smaller opening than the conductive sheath, the embodiment 201 (when deflated) can allow both E1 and E2 to cross the opening and be advanced further into the lesion, thus a larger area of the calcified lesion to be broken down in the first stage of the treatment and allowing the procedure to be completed more efficiently.

With reference to FIG. 2A, an elongated tube 204 has a tapered distal portion. All wires are tacked down using adhesive. A layer of polymer, such as polyethylene terephthalate (“PET”), can cover the wires before and after the emitters/insulation removed spots to hold down the wires. A number of insulated wires (e.g., lead or interstation wires) 230, 234, 236, and 238 are disposed on the outer surface of the elongated tube 204 such that they extend along the length of the elongated tube. The wires can be straight or twisted. Further, two conductive sheaths 212 and 214 each circumferentially mounted around the elongated tube 204.

FIG. 2B depicts the relative placement of the wires and the conductive sheaths, with the elongated tube removed. FIG. 2B further depicts two current flows 2a and 2b for generating six sources of shock waves, as described with reference to FIGS. 2C and 2D.

FIGS. 2C and 2D schematically depict two electrical diagrams of the configuration of FIGS. 2A-B, in accordance with some embodiments. With reference to FIG. 2C, when a first high voltage is applied (e.g., using the high voltage pulse generator 150 of FIG. 1B) across the proximal end of the insulated wire 230 and the proximal end of the insulated wire 234, a current 2a may flow as indicated by the arrows, with the insulated wire 230 as the common ground wire (i.e., connecting to a ground or negative channel). As shown, the current flows from the proximal end of the insulated wire 234 toward to the distal end of the insulated wire 234 and, via the insulation removed spot that is electrically conductive on wire 234 (i.e., electrode 234a), to the insulation removed spot that is electrically conductive on the proximal end of wire 238 (i.e., electrode 238a, generating shock waves (“SW1”). The shockwave propagates and extends outward from the 2 insulation removed spots 234a and 238a.

The current 2a may further traverse toward the distal end of wire 238 and, via the insulation removed spot near the distal end of the insulated wire 238 (i.e., electrode 238b), to the insulation removed spot near the distal end of the insulated wire 230 (i.e., electrode 230a), generating shock waves (“SW2”). The shockwave propagates and extends outward from the 2 insulation removed spots 238b and 230a. The current 2a then returns to the voltage source generator via the insulated wire 230 to a voltage output port (not depicted), which may be a negative channel or a ground channel. Optionally, a connector (not depicted) may be provided between the insulated wires 234 and 230 and the voltage pulse generator so that the wires may be easily connected to the output ports of the high voltage generator.

With reference to FIG. 2D, when a second high voltage is applied (e.g., using the high voltage pulse generator 150 of FIG. 1B) across the proximal end of the insulated wire 230 and the proximal end of the insulated wire 232, a current 2b may flow as indicated by the arrows, with the insulated wire 230 as the common ground wire (i.e., connecting to a ground or negative channel). As shown, the current flows from the proximal end of the insulated wire 232 toward to the distal end of the insulated wire 232 and, via the insulation removed spot that is electrically conductive (i.e., inner electrode 232a), to the distal side edge of the conductive sheath 212 (i.e., outer electrode), generating shock waves (“SW3”). The current 2b may further traverse from the proximal side edge of the conductive sheath 212 (i.e., outer electrode) to the insulated wire 236, via the insulation removed spot near the proximal end of the insulated wire 236 (i.e., inner electrode 236a), generating shock waves (“SW4”). As shown, 232a and 236a are positioned circumferentially 180 degrees from each other on opposite edges of the conductive sheath 212.

The current 2b may further traverse toward the distal end of the wire 236 and, via the insulation removed spot that is electrically conductive near the distal end of the wire 236 (i.e., the electrode 236b), to the proximal side edge of conductive sheath 214, generating shock waves (“SW5”). The current 2b may further traverse from the distal side edge of the conductive sheath 214) to the insulated wire 230, via the insulation removed spot on the insulated wire 230 (i.e., inner electrode 230b), generating shock waves (“SW6”). The current then returns to the voltage source generator via the insulated wire 230 to a voltage output port. As shown, 236b and 230b are positioned circumferentially 180 degrees from each other on opposite edges of conductive sheath 214.

The current flows depicted in FIGS. 2C and 2D can occur simultaneously (e.g., by applying two voltage channels simultaneously) or at different times depending on the stage of the treatment. In the exemplary treatment discussed above, only current 2a is activated during the first stage of the treatment, thus activating only SW1, and SW2. This is to break loose calcifications in a tight lesion that only the distal segments (e.g., distal cone and/or distal leg) of the balloon can cross or come close to. After calcium in the tight lesion has been modified, the balloon is deflated and advanced further into the lesion. The balloon is again inflated to IVL pressure. In the second stage of the IVL treatment, all of the emitters are activated simultaneously as shown in FIGS. 2C and 2D. Shock waves generated by the proximal emitters (i.e., SW3, SW4, SW5, SW6) provide additional treatment to the calcific lesion, while shock waves generated by the two distal emitters (i.e., SW1, SW2) allows further advancement of the balloon. Alternatively, only SW3, SW4, SW5, and SW6 are activated in the 2^{nd} stage.

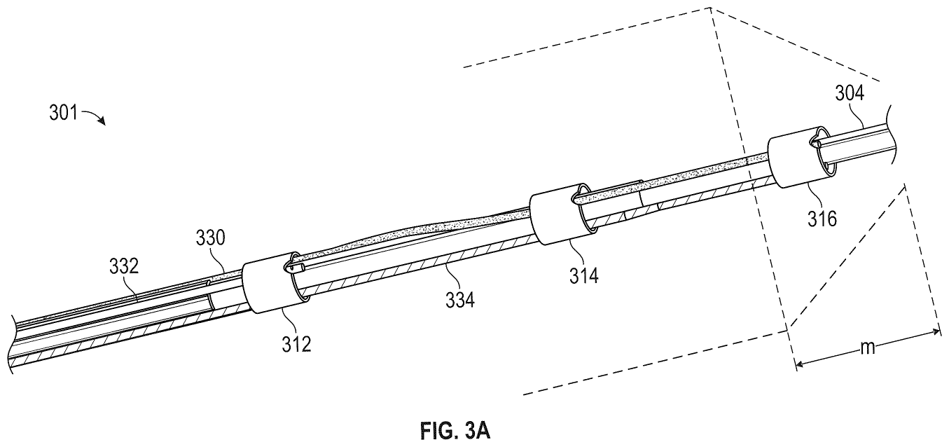

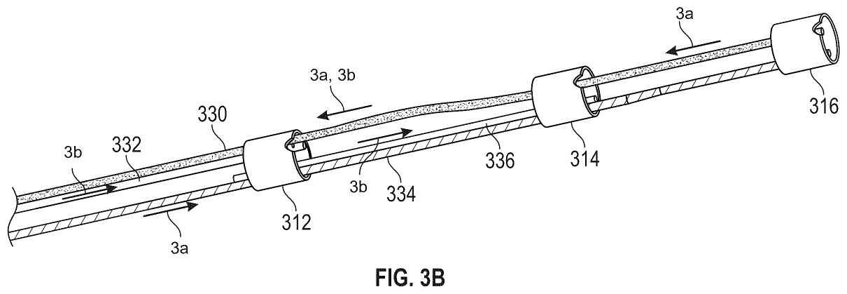

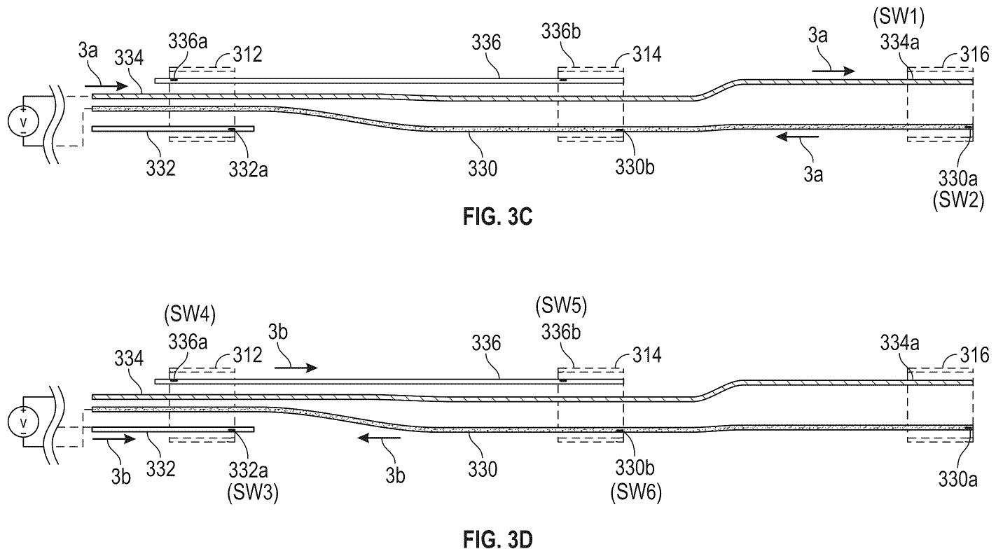

FIGS. 3A-D depict another exemplary shock wave electrode assemblies 301 that can be included in the exemplary shock wave angioplasty device 100 of FIG. 1B, according to some embodiments of the invention. Unlike embodiment 101 and embodiment 201, the embodiment 301 does not include any wire-to-wire electrode pairs. Rather, all emitters are formed by a side edge of a conductive sheath and a conductive portion of a wire. Further, the two most distal emitters (formed at the two side edges of the conductive sheath 316) are both disposed in the cone segment (“M”) of a balloon.

With reference to FIG. 3A, a number of insulated wires (e.g., lead or interstation wires) 330, 332, 334 and 336 are disposed on the outer surface of the elongated tube 304 such that they extend along the length of the elongated tube. The wires can be straight or twisted. Further, three conductive sheaths 312, 314, and 316 each circumferentially mounted around the elongated tube 304.

FIG. 3B depicts the relative placement of the wires and the conductive sheaths, with the elongated tube removed. FIG. 3B further depicts two current flows 3a and 3b for generating six sources of shock waves, as described with reference to FIGS. 3C and 3D.

FIGS. 3C and 3D schematically depict two electrical diagrams of the configuration of FIGS. 3A-B, in accordance with some embodiments. With reference to FIG. 3C, when a first high voltage is applied (e.g., using the high voltage pulse generator 150 of FIG. 1B) across the proximal end of the insulated wire 330 and the proximal end of the insulated wire 334, a current 3a may flow as indicated by the arrows, with the insulated wire 330 as the common ground wire (i.e., connecting to a ground or negative channel). As shown, the current flows from the proximal end of the insulated wire 334 toward to the distal end of the insulated wire 334 and, via the insulation removed spot that is electrically conductive on wire 334 (i.e., electrode 334a), to the proximal side edge of the conductive sheath 316, generating shock waves (“SW1”).

The current 3a may further traverse from the distal side edge of the conductive sheath 316 and, via the insulation removed spot near the distal end of the insulated wire 330 (i.e., electrode 330a), to the wire 330, generating shock waves (“SW2”). The current 3a then returns to the voltage source generator via the insulated wire 330 to a voltage output port (not depicted), which may be a negative channel or a ground channel. Optionally, a connector (not depicted) may be provided between the insulated wires 334 and 330 and the voltage pulse generator so that the wires may be easily connected to the output ports of the high voltage generator.

With reference to FIG. 3D, when a second high voltage is applied (e.g., using the high voltage pulse generator 150 of FIG. 1B) across the proximal end of the insulated wire 330 and the proximal end of the insulated wire 332, a current 3b may flow as indicated by the arrows, with the insulated wire 330 as the common ground wire (i.e., connecting to a ground or negative channel). As shown, the current flows from the proximal end of the insulated wire 332 toward to the distal end of the insulated wire 332 and, via the insulation removed spot that is electrically conductive (i.e., inner electrode 332a), to the distal side edge of the conductive sheath 312 (i.e., outer electrode), generating shock waves (“SW3”). The current 2b may further traverse from the proximal side edge of the conductive sheath 312 (i.e., outer electrode) to the insulated wire 336, via the insulation removed spot near the proximal end of the insulated wire 336 (i.e., inner electrode 336a), generating shock waves (“SW4”). As shown, 332a and 336a are positioned circumferentially 180 degrees from each other on opposite edges of the conductive sheath 312.

The current 3b may further traverse toward the distal end of the wire 336 and, via the insulation removed spot that is electrically conductive near the distal end of the wire 336 (i.e., the electrode 336b), to the proximal side edge of conductive sheath 314, generating shock waves (“SW5”). The current 3b may further traverse from the distal side edge of the conductive sheath 314) to the insulated wire 330, via the insulation removed spot on the insulated wire 330 (i.e., inner electrode 330b), generating shock waves (“SW6”). The current then returns to the voltage source generator via the insulated wire 330 to a voltage output port. As shown, 336b and 330b are positioned circumferentially 180 degrees from each other on opposite edges of conductive sheath 314.

The current flows depicted in FIGS. 3C and 3D can occur simultaneously (e.g., by applying two voltage channels simultaneously) or at different times depending on the stage of the treatment. In the exemplary treatment discussed above, only current 3a is activated during the first stage of the treatment, thus activating only SW1, and SW2. This is to break loose calcifications in a tight lesion that only the distal segments (e.g., distal cone and/or distal leg) of the balloon can cross or come close to. After calcium in the tight lesion has been modified, the balloon is deflated and advanced further into the lesion. The balloon is again inflated to IVL pressure. In the second stage of the IVL treatment, all of the emitters are activated simultaneously as shown in FIGS. 3C and 3D. Shock waves generated by the proximal emitters (i.e., SW3, SW4, SW5, SW6) provide additional treatment to the calcific lesion, while shock waves generated by the three distal emitters (i.e., SW1, SW2) allows further advancement of the balloon.

It will be understood that the foregoing is only illustrative of the principles of the invention, and that various modifications, alterations and combinations can be made by those skilled in the art without departing from the scope and spirit of the invention. Any of the variations of the various shock wave devices disclosed herein can include features described by any other shock wave devices or combination of shock wave devices herein. Furthermore, any of the methods can be used with any of the shock wave devices disclosed. Accordingly, it is not intended that the invention be limited, except as by the appended claims. For all of the variations described above, the steps of the methods need not be performed sequentially

1. A method of generating shock waves, the method comprising:
introducing a shock wave device into a patient's vasculature, the shock wave device comprising:
a balloon comprising:
a distal end;
a leg segment proximal to the distal end of the balloon;
a cone segment proximal to the leg segment of the balloon; and
a straight segment proximal to the cone segment of the balloon;
at least one distal emitter in the leg segment or the cone segment of the balloon; and
at least one proximal emitter in the straight segment of the balloon; during a first stage:
advancing the shock wave device in the patient's vasculature such that the distal end of the balloon is advanced into a calcified lesion;
inflating the balloon with a conductive fluid such that the inflated balloon is gently fixed to walls of the vasculature in direct proximity with the calcified lesion; wherein, when the balloon is inflated, the leg segment is substantially cylindrical or is of a tapered shape with an average diameter, the cone segment is of a tapered shape with an average diameter greater than the average diameter of the leg segment, and the straight segment is of a substantially cylindrical shape with a diameter greater than the average diameter of the cone segment;
activating a voltage source to generate shock waves at the at least one distal emitter; and
after activating the voltage source, deflating the balloon; and
during a second stage:
further advancing the shock wave device in the patient's vasculature such that the straight segment of the balloon is advanced into the calcified lesion;
inflating the balloon with the conductive fluid such that the inflated balloon is gently fixed to walls of the vasculature in direct proximity with the calcified lesion; and
activating the voltage source to generate shock waves at the at least one proximal emitter.

2. The method of claim 1, wherein the first stage is repeated until the calcified lesion is modified such that the straight segment of the balloon is able to cross the calcified lesion.

3. The method of claim 1, wherein activating the voltage source during the second stage generates shock waves at both the at least one proximal emitter and the at least one distal emitter.

4. The method of claim 1, wherein the at least one distal emitter is positioned in the leg segment of the balloon.

5. The method of claim 1, wherein the at least one distal emitter is positioned in the cone segment of the balloon.

6. The method of claim 1, wherein the at least one distal emitter comprises a first distal emitter positioned in the leg segment of the balloon and a second distal emitter positioned in the cone segment of the balloon.
